(19) United States
(12) Patent Application Publication
(10) Pub. No.: US 2025/0108075 A1
(43) Pub. Date: Apr. 3, 2025

(54) METHODS OF ADMINISTERING COMPOSITIONS OF BACILLUS SUBTILIS PB6, BUTYRIC ACID WITH ZINC, AND OPTIONALLY RED CLOVER, TO REDUCE LIVER ABSCESSES IN CATTLE

(71) Applicant: KEMIN INDUSTRIES, INC., Des Moines, IA (US)

(72) Inventor: Jerilyn Elena Hergenreder, Maxwell, IA (US)

(21) Appl. No.: 18/902,290

(22) Filed: Sep. 30, 2024

Related U.S. Application Data
(60) Provisional application No. 63/541,468, filed on Sep. 29, 2023.

(51) Int. Cl. A61K 35/742; A23K 10/18; A23K 10/30; A23K 20/105; A23K 20/20; A23K 50/10; A61K 31/19; A61K 33/30; A61K 36/48; A61P 1/16

(52) U.S. Cl. CPC A61K 35/742; A23K 10/18; A23K 10/30; A23K 20/105; A23K 20/30; A23K 50/10; A61K 31/19; A61K 33/30; A61K 36/48; A61P 1/16

(57) ABSTRACT

The present invention relates to administering an animal feed additive or supplement that contains a combination of Bacillus subtilis PB6, butyric acid and zinc, and optionally red clover, in an amount effective to decrease the incidence and severity of liver abscesses in weaned beef and dairy steers.

### CROSS-REFERENCE TO RELATED APPLICATIONS

The present application claims the benefit of priority to U.S. Provisional Patent Application No. 63/541,468, entitled “METHODS OF ADMINISTERING COMPOSITIONS OF Bacillus subtilis PB6, BUTYRIC ACID WITH ZINC, AND OPTIONALLY RED CLOVER, TO REDUCE LIVER ABSCESSES IN CATTLE,” filed Sep. 29, 2023, the entire disclosure of which is incorporated by reference herein.

### BACKGROUND OF THE INVENTION

Liver abscesses are a common and costly condition in the fed cattle industry, affecting approximately 12 to 32% of finished cattle (Amachawadi and Nagaraja, 2016). Severe abscesses have been linked to decreased growth performance, leading to appreciable economic losses for cattle feeders and beef processors. Beef livers are an export variety meat, and the U.S. exported 76% of beef livers to Egypt (62,119 metric tons) in 2016 (Global Trade Atlas, 2017). The presence of a liver abscesses (LA) can decrease the value of beef carcasses by $38 per animal (Brown and Lawrence, 2010) and cost the cattle feeder and beef processor >$470 million dollars annually (Herrick et al., 2022). Therefore, effective prevention and control strategies are needed to decrease the economic burden associated with LA.

Two bacterial species are likely associated with the development of LA in cattle, Fusobacterium necrophorum, and Salmonella enterica serovar Lubbock (Amachawadi and Nagaraja, 2015). Although the role of S. enterica. in LA formation is not fully understood, it has been used in combination with F. necrophorum to successfully induce LA (McDaniel et al., 2023). Indeed, if S. enterica contributes to the development of LA, supplementation with Bacillus subtilis PB6 could decrease LA prevalence in cattle because it has been reported to decrease Salmonella in the feces of cattle (Broadway et al., 2020; Smock et al., 2020).

Leaky gut could also be a contributing factor to LA formation, but it is unclear whether pathogens invade the liver through ruminal lesions or leaky tight junctions in the lower gastrointestinal tract (Sanz-Fernandez et al., 2020). Assuming the latter, butyric acid and zinc supplementation might improve tight junction integrity and barrier function in the lower gastrointestinal tract by increasing villus height, crypt depth, and maintaining osmotic pressure (Ohata et al., 2005).

Tylosin phosphate, a macrolide antibiotic, is currently administered in commercial feedlots to reduce incidences of liver abscesses, though it has been reported that there is variable efficacy among different groups of cattle. (Weinroth et al., 2019). Additionally, growing concerns of antibiotic resistance has prompted urgency in finding alternative solutions for decreasing the incidence and severity of liver abscesses in cattle. For instance, the United States Food and Drug Administration (FDA) banned the use of antibiotics as growth promoters in cattle feed and drinking water in 2015. These products can only be used for therapeutic purposes under veterinary oversight (Veterinary Feed Directive, 2015). Due to consumer pressure and this regulation, finding a suitable alternative to tylosin phosphate has been a topic of critical research (Rode et al., 2002; Beauchemin and McGinn, 2006; Dehghani et al., 2019).

### BRIEF DESCRIPTION OF THE INVENTION

The present invention relates to administering a feed additive or supplement to cattle that is a combination of Bacillus subtilis PB6, butyric acid and zinc, and optionally red clover, to decrease the incidence and severity of liver abscesses in weaned beef and dairy steers. Another aspect of the present invention relates to administering the feed additive or supplement containing a combination of Bacillus subtilis PB6, butyric acid and zinc to Bos Taurus influenced natural fed cattle in an amount effective to decrease the incidence of liver abscesses.

### BRIEF DESCRIPTION OF THE FIGURES

FIG. 1 depicts the daily feed disappearance for beef×dairy steers fed ground corn with or without (CON) Bacillus subtilis and encapsulated butyric acid and zinc (CV), and CV+red clover (Trifolium pratense; CVC) to evaluate the formation of liver abscesses. Error bars denote the standard errors of least squares means (n=10 to 15 steers/mean). Treatment, time, and treatment×time (P<0.01). * Denotes treatment and time difference P<0.01.

### DETAILED DESCRIPTION OF THE INVENTION

The present invention relates to administering a combination of Bacillus subtilis PB6 and butyric acid with zinc, with or without red clover or extracts, to decrease the incidence and severity of liver abscesses in weaned beef and dairy steers.

According to at least one embodiment, a combination of Bacillus subtilis PB6 and butyric acid with zinc is administered to cattle in an amount to reduce the incidence or severity of LA in cattle. In at least one embodiment, the following ingredients were administered: B. subtilis PB6 (CLOSTAT Dry® dry, 2.2×10⁸ CFU/g) in an amount ranging from between about 0.5 g to about 15 g/steer daily, for instance 13 g/steer daily, butyric acid in an amount ranging from about 0.5 g to about 5 g/steer daily, and zinc in an amount ranging from about 0.1 to about 0.5 g daily, and in certain embodiments, optionally up to 30 g red clover daily.

According to at least one embodiment, a combination of Bacillus subtilis PB6 and butyric acid with zinc is administered to cattle in order to improve beef carcass scores. In at least one embodiment, the following ingredients were administered: B. subtilis PB6 (CLOSTAT Dry® dry, 2.2×10⁸ CFU/g) in an amount ranging from between about 0.5 g to about 15 g/steer daily, for instance 13 g/steer daily, butyric acid in an amount ranging from about 0.5 g to about 5 g/steer daily, and zinc in an amount ranging from about 0.1 to about 0.5 g daily, and in certain embodiments, optionally up to 30 g red clover daily.

In at least one embodiment, the butyric acid and zinc is encapsulated, where the cattle were fed encapsulated butyric acid with zinc (3 g/daily) to deliver a dosage of about 0.75 g butyric acid and 0.3 g zinc. In alternative embodiments, the butyric acid and zinc may be delivered in alternative forms, with or without encapsulation. Persons of ordinary skill in the art would appreciate that the dosage may vary based on the specific circumstances on the feedlot, for instance in certain embodiments, the dosage of butyric acid and zinc may be increased or decreased. In alternative embodiments, the dosage of PB6 may be increased or decreased. In certain embodiments, the additive or supplement combination further comprises red clover or an extract of red clover.

According to at least one embodiment, the combination is administered to cattle at least once a day for at least 25 days before harvest. In alternative embodiments, the composition is administered to cattle between about 25 days to about 100 days, for instance for at least 35 days, at least 40 days, or at least 45 days, at least 50 days, at least 55 days, at least 60 days, at least 65 days, at least 70 days, at least 75 days, at least 80 days, at least 85 days, at least 90 days, at least 95 days, and at least 100 days before harvest. According to at least one embodiment, the combination is administered to the cattle between days 150 to 250 days on feed.

In at least one embodiment, the combination is administered to cattle for at least 35 days before harvest. In at least one embodiment, the combination is administered to cattle for at least 50 days before harvest. In another embodiment, the combination is administered to cattle for at least 65 days before harvest. In another embodiment, the combination is administered to cattle for at least 75 days before harvest.

In alternative embodiments, the combination is administered to the cattle at least once daily for at least 25 days before the cattle is harvested, wherein the administration of the feed additive or supplement occurs daily between days 100 to 300 days on feed, for instance between days 150 to 250 or days 170 to 200.

In at least one embodiment, the combination is administered orally at least once daily. In alternative embodiments, the combination is administered twice a day. In alternative embodiments, the combination is administered up to three times a day.

### Example 1

Materials and Methods: Forty beef×dairy steers (initial BW 97±11.5 kg) were individually housed in a climate-controlled barn. Steers were assigned randomly to 1 of 3 treatments: 1) cycled between a low-starch control and high-starch acidotic diet (CON; n=10); 2) cycled between the control and high-starch acidotic diet and supplemented with 13 g/steer daily of B. subtilis PB6 (CLOSTAT Dry® dry, 2.2×10⁸ CFU/g, Kemin Industries, Inc., Des Moines, IA)+3 g/steer daily of butyric acid (0.75 g daily) and zinc (0.3 g daily; VilliTech, Kemin Industries, Inc; CV; n=15); or 3) CV+30 g/steer daily of chopped red clover (CVC; n=15). All steers were intraruminally inoculated with Fusobacterium necrophorum subsp. necrophorum and Salmonella enterica serovar Lubbock on d 20. After euthanasia on d 39, necropsies were performed to assess gross pathology.
Results and Discussion: Steers in CVC had a greater percentage of normal lung scores than steers in the CON in CV treatments (P=0.04). Liver abscess prevalence did not differ among treatments (P=0.68); however, liver abscess prevalence decreased 25% in CV vs. CON and 22% in CVC vs. CON. Feed disappearance decreased in all treatments from d 0 to 3 and from d 20 to 22 but otherwise increased over time. Steers in CV consumed more feed than CON and CVC from d 24 to 38 (P<0.01).
Implications and Applications: Our data demonstrate the viability of the experimental model to evaluate LA mitigation strategies. Although no statistical differences in LA were attributable to the supplements evaluated, the decrease in liver abscess prevalence we noted with CV could be biologically and economically impactful. Further research is needed to elucidate specific mechanisms of abscess prevention via nutritional supplementation.

All experimental procedures involving live animal subjects were approved by the Livestock Issues Research Unit Institutional Animal Care and Use Committee (approval number: 2023-02).

### Animal Management and Experimental Treatment Application

Weaned beef×dairy steers (n=40; initial BW=97±11.5 kg) were obtained from a commercial calf ranch in eastern New Mexico. Steers were transported to the USDA-ARS Livestock Issues Research Unit facilities near Lubbock, TX on d −22 relative to the first diet cycle (Table 1). On arrival, steers were individually weighed to nearest 0.2 kg (MP600 load cells; Tru-Test Group, Auckland, New Zealand), given an ear tag with unique identification, dewormed with fenbendazole (Safeguard, Merck Animal Health, Whitehouse Station, NJ), and administered tildipirosin as a prophylactic antibiotic for the prevention of respiratory disease (Zuprevo, Merck Animal Health).

TABLE 1
Timeline of key events in which beef × dairy steers
were fed a low-starch control diet (CON) and then cycled
to a high-starch acidotic diet. Each diet included ground corn
with or without Bacillus subtilis and encapsulated butyric acid
and zinc (CV) or CV + red clover (Trifolium pratense; CVC)
to evaluate the formation of liver abscesses¹.

| Item | Day |
|---|---|
| Adaptation to facility | −22 to −18 |
| Steers fed basal diet | −22 to −1 |
| Acidotic cycles | |
| Cycle 1 | 0 to 2 |
| Cycle 2 | 5 to 7 |
| Cycle 3 | 10 to 12 |
| Cycle 4 | 15 to 17 |
| Continuous feeding of acidotic diet | 18 |
| Body weight measurement | −16, −9, −2, 5, 12, 19, 26, 33, 38 |
| Bacterial inoculation | 20 |
| Blood collection | −2, 12, 26, 38 |
| Harvest and gross pathology | 39 |

¹Between cycles 1, 2, and 3, steers remained on the control diet for 2 d. For cycle 4, ahead of inoculation, steers remained on the control diet for 3 d to ensure ruminal pH would not lyse the inoculated F. necrophorum. The bacterial inoculation included a mixture of Fusobacterium necrophorum (8.00 × 10⁸ CFU), and Salmonella enterica (2.67 × 10⁹ CFU/mL).

Steers were assigned randomly to 1 of 3 experimental treatments balanced by BW: 1) steers were cycled between a low-starch control and high-starch acidotic diet (CON; n=10); 2) steers were cycled between a low-starch control and high-starch acidotic diet and supplemented with 13 g/steer daily of B. subtilis PB6 (CLOSTAT® Dry, Kemin Industries, Inc., Des Moines, IA)+3 g/steer daily of butyric acid and zinc (VilliTech, Kemin Industries, Inc; CV; n=15); and 3) steers were cycled between a low-starch control and high-starch acidotic diet and as supplemented as with the CV treatment with the addition of 30 g/steer daily of chopped red clover (CVC; n=15; Monterey Bay Herb Company, Watsonville, CA). All supplements were mixed into a ground corn carrier twice during the study (d −22 and 10) and 115 g of the supplement was top dressed and mixed with the diet at the time of feeding at approximately 0800 h. Steers in the CON treatment group received an equivalent amount of ground corn with no added supplement.

Diets were formulated to meet nutrient requirements for growing cattle (NASEM, 2016). Ingredient and analyzed nutrient composition of the diets are summarized in Table 2. Before d 0, all steers were fed the CON diet for 22 d. The diet was offered at 2.5% of BW on a DM basis on the day of arrival, after which steers were fed to appetite targeting ad libitum intake with ad libitum access to fresh water. The diets were formulated to provide different concentrations of starch and NDF. The control diet was a low-starch growing diet, whereas the acidotic diet was formulated to have more fermentable starch and less in NDF to promote acidotic conditions in the rumen. In all treatments, on d 0, the diet was switched from the control to acidotic diet for 3 d, followed by feeding control diet for 2 d. This 5-d cycle was repeated 4 times during the study. Weekly diet samples were obtained to form a weekly composite for analyses of CP, NDF, total starch, crude fat, Ca, and P contents (Servi-Tech Laboratories, Amarillo, TX). A second diet sample was dried at 100° C. for 24 h in a forced-air oven (Thermo Scientific Heratherm OGS100, Thermo Fisher Scientific, Inc., Waltham, MA) for analysis of DM to calculate DMI. Steers were housed on a slatted floor, so data presented represents feed disappearance and not feed intake because a small amount might have fallen through the slats. Feed disappearance was recorded as DM fed minus DM of the orts. Daily feed deliveries and orts were weighed to the nearest 5 g at 0800 h (UWE AMP-150 NTEP bench scale; Intelligent Weighing Technology, Inc. Democratic Taiwan). In the event of bloat, a flexible tube (0.95 cm internal diameter×1.5 m long) was passed orally into the rumen to relieve pressure. Bloat was relieved for 5 steers throughout the study (2 from CON, 1 from CV, and 2 from CVC; data not presented).

On day 20, steers were gently restrained in a squeeze chute to allow for the collection of ruminal fluid and fecal samples. Additionally, steers were administered the bacterial inoculation described below while they were restrained. For collection of ruminal fluid and the bacterial inoculation, a flexible tube 1.91-cm diameter was passed through a frick-type speculum from the oral cavity to the level of the oropharynx and directed to the esophagus and passed into the rumen. The bacterial inoculation contained ˜100 mL each of S. enterica (2.67×10⁹ CFU/g) and F. necrophorum (8.00×10⁸ CFU/g). Following inoculation, the flexible tube was flushed with 100 mL of phosphate buffered saline to clear any residual inoculant. After the bacterial inoculation on day 20, steers were fed the acidotic diet for the remainder of the study. Body weights and rectal temperatures were measured on d −16, −9, −2, 5, 12, 19, 26, 34 and at harvest on day 39.

TABLE 2
Ingredient and nutrient composition of the low-starch
control and acidotic diets fed to beef × dairy
steers to evaluate the formation of liver abscesses¹

Diet

| Item | Control | Acidotic |
|---|---|---|
| Ingredient, % of dry matter (DM) | | |
| Steam-flaked corn | — | 54.40 |
| Dry-rolled corn | 20.20 | — |
| Wet corn gluten feed² | 46.10 | 21.90 |
| Ground corn | — | 14.90 |
| Ground alfalfa hay | 30.20 | 4.60 |
| Limestone | 1.50 | 1.50 |
| Urea | — | 0.70 |
| Vitamin and mineral supplement³ | 2.00 | 2.00 |
| Analyzed nutrient composition⁴ | | |
| Diet DM, % | 72.30 | 77.90 |
| Crude protein, % | 18.60 | 16.80 |
| Neutral detergent fiber, % | 35.00 | 15.10 |
| Total starch, % | 20.70 | 57.40 |
| Crude fat, % | 2.90 | 3.00 |
| Ca, % | 0.93 | 0.66 |
| P, % | 0.61 | 0.36 |
| NEₘ⁵, Mcal/kg | 1.67 | 2.29 |
| NE_{g}⁵, Mcal/kg | 1.08 | 1.58 |

¹Dry matter basis, except diet DM %.
²SweetBran; Cargill, Inc. Blair, NE.
³Supplement supplied 44.40% crude protein, 5.99% potassium chloride, 3.82% salt, 8.34 mg/kg cobalt carbonate, 395.0 mg/kg copper sulfate, 408.0 mg/kg iron sulfate, 764 mg/kg manganous oxide, 2.92 mg/kg sodium selenate, and 2,490.00 mg/kg zinc sulfate on a DM basis.
⁴Analysis performed by Servi-Tech Laboratories, Amarillo, TX.
⁵NEₘ and NE_{g} are tabular values (NASEM, 2016).

Each morning throughout the experiment, trained personnel visually monitored steers for clinical signs of illness using a system described by Step et al. (2008) and Wilson et al. (2015). Throughout the study, 25 steers were given additional antibiotic therapy, which included 9 steers from CON, 5 steers from CV, and 11 steers from CVC. One steer from CON and one steer from CVC were euthanized for chronic respiratory illness on days 20 and 24, respectively.

### Gross Pathology Scores

All steers were humanely harvested on day 39. At necropsy, gross pathology was recorded on the lungs, rumens, livers, and colons. The lung scoring system developed by Tennant et al. (2014) was used, in which lungs were scored as normal, mild, or severe depending on whether there were visible or palpable lesions associated with bovine respiratory disease (BRD) or mild hyperemia of the cranioventral lobes without consolidation. A mild score indicated lesions associated with BRD in up to 50% of the cranioventral lobe(s). Steers with severe scores had lesions associated with BRD and pleural adhesions within the thoracic cavity or consolidation of 51 to 100% of their cranioventral lobes.

Rumens were scored as normal, mild, and severe. A normal score indicated healthy epithelium without ulceration or inflammation, a mild score indicated consolidation of ruminal mucosal surface or denuded papillae, and a severe score indicated active rumenitis lesions or ulcers. Livers were scored as either normal or abnormal, with a score of 0 indicating no detectable abscess and a score of A− indicating at least 1 detectable abscess, A indicating at least 2 abscesses and depending on the size and severity of the abscess, and A+ indicating at least 3 liver abscesses and showing the most severe cases of liver abscesses (Brown et al., 1975).

Colons were scored on a scale from 1 to 4, in which a score of 1 indicated a colon free of parasites and bacteria according to Mansfield and Urban (1996). Scores of 2 indicated patchy hemorrhage (50% is hemorrhagic) and slightly roughened tissue. A score of 3 indicated severe hemorrhage (more than 50% of surface area) and noticeable roughness. A score of 4 indicated the colons were covered in fibrinonecrotic pseudomembrane, with sloughed mucosa and lymphoglandular complexes.

### Liver Abscess Culturing Techniques

At necropsy, if a LA was identified, it was aseptically removed from the liver, placed on ice, and shipped overnight to Kansas State University College of Veterinary Medicine to determine the prevalence of F. necrophorum and S. enterica. Culture procedures for F. necrophorum and S. enterica were conducted as described by Amachawadi et al. (2017). Briefly, a sterile scalpel was used to cut the capsule of abscesses after they were seared with a hot spatula. To isolate S. enterica, the inner wall of the abscess capsule was swabbed and streaked onto three plates blood agar (Remel) and 2 plates of Hektoen Enteric (HE) agar (Becton, Dickinson and Company). As a control, one HE and one blood agar plate were incubated aerobically within an aerobic glove box (Thermo Fisher Scientific Inc.), and a separate HE plate and blood agar plate were incubated anaerobically within an anaerobic glove box (Thermo Fisher Scientific Inc.). The Salmonella was enriched using tetrathionate broth (Becton Dickinson and Company; for 24 h at 37° C.) and Rappaport Vassiliadis broth (Becton, Dickinson and Company) for 24 h at 37° C. before isolation on HE agar (incubated 24 h at 37° C.). Presumptive, phenotypic colonies were agglutinated with S. enterica polyvalent O antiserum (Becton, Dickinson and Company) allowing for genus confirmation. On confirmation of positive results, B, C1, C2, D1, D2, and E antisera were tested for serogroup classification. Brain heart infusion broth that was pre-reduced with 0.05% cysteine HCl and anaerobically sterilized (BHI-S) was inoculated with presumed F. necrophorum from an abscess. In addition to microscopic morphology, Rapid-ANA II test kits (Thermo Fisher Scientific Inc.) were used for confirmation of the subspecies (necrophorum or funduliforme) determined by sedimentation characteristics in phosphate test and BHI-S broth (Tan et al., 1994b).

### Sample Processing for Salmonella Prevalence

Fecal samples were collected on days-18, 24, 38, and subiliac and mesenteric lymph nodes, and ileum were collected at necropsy. To determine the prevalence of Salmonella, a 25-g sample was mixed with a 1:10 dilution of PBS in a special bag (Seward Ltd.; West Sussex, UK), after which it was blended using a Stomacher® 400 Circulator (Seward Ltd.) for 2 min at 230 rotations per min. Before blending, subiliac lymph nodes were crushed with a rubber mallet (Arthur et al., 2008). The bags were sealed, gently massaged by hand, and 1 mL of the mixture was enriched by diluting it 1:10 with Tetrathionate Hajna broth (Remel Inc., San Diego, CA) containing iodine and Rappaport-Vassiliadis broth (Oxoid Ltd., Basingstoke, UK). These enriched mixtures were then placed in an incubator, with some at 37° C. and others at 42° C. and left overnight. After incubation, the enriched cultures were mixed, and a small 10-μL loop was used to streak the enriched material onto xylose lysine deoxycholate (XLD; Becton, Dickinson and Co., Franklin Lakes, NJ) agar that had novobiocin (25 μg/mL) added. These streaked agar plates were then incubated at 37° C. overnight and an additional 24 h at 25° C. Finally, colonies that looked like Salmonella were confirmed using latex agglutination (Salmonella Test Kit; Oxoid Ltd).

### Quantitative Polymerase Chain Reaction and Tissue Culture Methods

Ruminal fluid and tissue, colon content and tissue, ileum tissue, healthy liver tissue, and liver abscesses (if present) were aseptically collected from a consistent anatomical location during necropsy. Samples were stored at 4° C. before being shipped overnight to Kansas State University for quantitative polymerase chain reaction (q-PCR) and culture tissues for determining the prevalence of F. necrophorum subsp. necrophorum and funduliforme within each individual sample.

Whole Genome Sequencing of the F. necrophorum Subsp. necrophorum Strains

Strains isolated from the liver abscesses and the ruminally inoculated strain F. necrophorum, 8L1 were cultured, genomic DNA was extracted, and samples submitted for whole genome sequencing to CosmosID (Germantown, MD). Following the manufacturer's protocol, genomic DNA was extracted using DNeasy Blood & Tissue Kit (Qiagen, Germantown, MD). Extracted DNA samples were quantified using the QuantiFluor® dsDNA System (Promega, Madison, WI) chemistry using GloMax Plate Reader Systems (Promega, Madison, WI). One nanogram of DNA was used to prepare DNA libraries using the Nextera XT DNA Library Preparation Kit (Illumina) and IDT Unique Dual Indexes. To fragment the genomic DNA, a proportional amount of the Illumina Nextera XT fragmentation enzyme was used. The samples were indexed with dual indices, and then PCR was performed for 12 cycles to create libraries. AMpure magnetic beads (Beckman Coulter) were used to purify DNA libraries, and QIAGEN EB buffer was used to elute samples. Qubit™ dsDNA HS Assay Kit and Qubit™ 4 fluorometer were used to quantify the DNA libraries. The libraries were sequenced at Cosmos ID Inc. (Rockville, MD) on an Illumina HiSeq X and NextSeq 2000 platform.

### Bioinformatics Analysis

The paired-end reads were processed and trimmed using the Genome Assembly tool provided by PATRIC (https://pubmed.ncbi.nlm.nih.gov/31667520/). Subsequently, single-nucleotide polymorphisms (SNP) from liver abscesses were identified and used to construct phylogenetic trees. The DNA sequences obtained from the NCBI GenBank, along with SNP found in the core genomes of the inoculated strains and strains isolated from liver abscesses were incorporated into the construction of phylogenetic trees. The maximum likelihood criterion was employed for the construction of these trees, ensuring accurate representation and classification of the strains (Allard et al., 2012).

Quantitative PCR Assay to Detect and Quantify F. necrophorum in Ruminal and Colonic Contents and Epithelial Tissues

The detection and quantification of F. necrophorum were determined using a real-time quantitative PCR assay targeting the subspecies-specific promoter sequences of the leukotoxin gene, IktA (Zhang et al., 2006).

### Statistical Analyses

Feed disappearance and BW was analyzed using PROC GLIMMIX of SAS (SAS Inst. Inc., Cary NC) as a repeated measures with individual steer as the experimental unit. The model included the fixed effects of treatment, time, and their interaction, with steer nested within treatment being the subject of repeated measures. Various covariate structures were tested, and the one resulting in the smallest Akaike and Schwarz Bayesian criteria was chosen for the analyses. The PROC GLIMMIX procedure was used to assess the differences in percent prevalence of Salmonella and liver abscesses and binomial proportions of necropsy scores among treatments. The model included the fixed effect of treatment. Both models included the Kenward-Rogers adjustment for an unequal number of experimental units among treatments. The significance level for all analyses was set at α=0.05, with statistical tendencies indicated at 0.05<P≤0.10. In cases where the main effects were significant, pairwise comparisons were performed (α=0.05) to examine differences among treatments.

### Results and Discussion

### Body Weight and Feed Disappearance

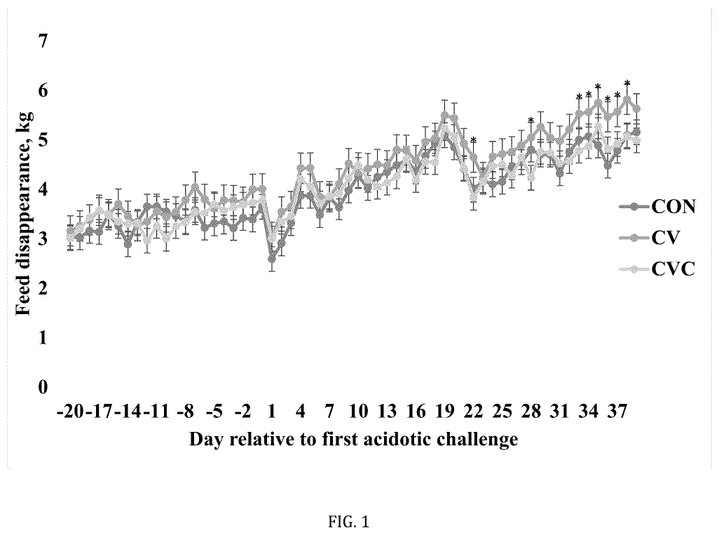

The were no differences in BW among treatments throughout the study (P=0.75; final BW=167, 175, and 170 kg±8.0 kg for CON, CV, and CVC, respectively; data not shown). According to Owens et al. (1998), when switching feedlot cattle from a high-forage diet to a high-concentrate diet, there can be a negative effect on the ruminal microbial populations that can decrease BW gain and increase metabolic disorders such as acidosis. Albornoz et al. (2013) and Zhang et al. (2013) also reported that decreased DMI followed by a return to ad libitum intake predisposes cattle to ruminal acidosis. For feed disappearance (FIG. 1), an interaction between treatment and time was observed (P<0.01), whereby feed disappearance was greater (P<0.01) in CV than CON and CVC on days 22, 28, and 33 to 38. In addition, feed disappearance was greater in CV and CVC than CON on d −6 to 0 (P<0.01). Steers in CON, CV, and CVC all had decreased feed intake from day 0 to 3 (P<0.01) and day 20 to 22 (P<0.01). From days 24 to 38, CV consumed more feed than CON or CVC (P<0.01). Our findings agree with Smith et al. (2021), who reported an increase in DMI when yearling crossbred beef steers were fed Bacillus subtilis. Broadway et al. (2020) reported increased DMI in weaned Holstein steers when supplemented with Bacillus subtilis PB6, which agrees with our results.

### Gross Pathology

The CVC steers had a greater proportion of normal lung scores than CON or CV (P=0.04; Table 3). Conversely, no differences were observed in the proportions of mild or severe lung scores among treatments (P≥0.15). Although CVC steers had the greatest proportion of normal lungs, 62.5% were therapeutically treated with tulathromycin for BRD relapse after the initial metaphylaxis was administered. The red clover included in the CVC supplement contains an isoflavone, biochanin A (Flythe et al., 2010; Harlow et al., 2017; Harlow et al., 2018). Biochanin A might have contributed to the increase in normal lung scores, as it has historically been valued as a respiratory remedy for whooping cough, measles, bronchitis, laryngitis, and tuberculosis in humans (Ko et al., 2011). Nonetheless, the concentration of biochanin A in the red clover was not measured in the current study. While it is important to note that all steers consumed all of their top-dressed supplement daily, future studies are necessary to better understand the effects of red clover and biochanin A on lung health in cattle.

TABLE 3
Gross pathological evaluation of tissues of beef × dairy
steers fed ground corn with or without (CON) Bacillus subtilis
and encapsulated butyric acid and zinc (CV), Bacillus subtilis,
encapsulated butyric acid and zinc, and CV plus red clover (Trifolium
pratense; CVC) cycled on an acidotic diet and inoculated with
Fusobacterium and Salmonella to evaluate liver
abscess formation and severity

| | Treatment¹ | | | | |
|---|---|---|---|---|---|
| Item | CON | CV | CVC | SEM² | P-value |
| Lung³, % | | | | | |
| Normal | 22.2^{a} | 6.7^{a} | 57.1^{b} | 14.00 | 0.04 |
| Mild | 66.7 | 80.0 | 42.9 | 15.70 | 0.15 |
| Severe | 11.1 | 13.3 | 0.0 | 10.51 | 0.99 |
| Rumen⁴, % | | | | | |
| Normal | 77.8 | 80.0 | 64.3 | 13.86 | 0.61 |
| Mild | 22.2 | 20.0 | 35.7 | 14.86 | 0.61 |
| Severe | 0.0 | 0.0 | 0.0 | — | 1.00 |
| Liver abscess⁵, | 77.8 | 60.0 | 64.3 | 13.86 | 0.68 |
| % | | | | | |
| 0 | 22.2 | 40.0 | 35.7 | 7.25 | 0.32 |
| A− | 0.0 | 0.0 | 0.0 | — | |
| A | 22.2 | 0.0 | 14.3 | 13.86 | 0.32 |
| A+ | 55.6 | 60.0 | 50.0 | 16.56 | 0.32 |
| No. of abscesses | 2.8 | 2.1 | 1.4 | 0.56 | 0.31 |
| per liver | | | | | |
| Colon⁶, % | | | | | |
| 1 | 77.8 | 66.7 | 85.7 | 13.86 | 0.50 |
| 2 | 22.2 | 33.3 | 14.3 | 13.86 | 0.50 |
| 3 | 0.0 | 0.0 | 0.0 | — | — |
| 4 | 0.0 | 0.0 | 0.0 | — | — |

^{a,b}Least squares mean in a row that do not have common superscripts differ; P < 0.05.
¹CON = control diet and acidotic diet with intraruminal bacterial inoculation; CV = control diet and acidotic diet supplemented with ground corn, CLOSTAT ® dry, and VilliTech ® with intraruminal bacterial inoculation; CVC = CV + red clover with intraruminal bacterial inoculation.
²Standard error of least squares means, n = 9 steers in CON; 15 steers in CV and 14 steers in CVC.
³Lung scored as normal, mild, and severe. Normal scores were lungs with no palpable lesions associated with BRD and no consolidation of cranioventral lobes. Severe scores were lungs with consolidation of 51 to 100% of cranioventral lobes, with lesions, and any adhesion to thoracic cavity (Thompson et al., 2006; Rezac et al., 2014).
⁴Ruminal epithelium scored as normal, mild, or severe. Normal scores indicate healthy epithelium, and lack of inflammation or ulceration. Severe scores indicate active rumenitis with inflammation and lesions.
⁵Liver abscess scored as 0 or abscessed. A score of 0 was a normal liver with no abscesses.
⁶Colons were scored as 1, 2, 3, or 4. A score of 1 indicated a healthy colon unaffected by parasites or bacteria; the gut was smooth and tan. A score of 4 indicated colons covered with fibrinonecrotic pseudomembrane, possibly denuded, and if not obliterated by pseudomembranes, filled with mucopurulent debris (Mansfield and Urban, 1996).

No differences among treatments were noted for ruminal scores (P=0.61). Among the 38 steers, 10 (CON=2, CV=3, and CVC=5) had signs of rumenitis. Rumenitis is characterized by inflammation of the ruminal epithelium and can be caused by various dietary factors (Nagaraja and Chengappa, 1998). In the current experiment, the acidotic diet fed and cycled to all treatments aimed to decrease ruminal pH rapidly, to attempt to induce rumenitis and subsequently facilitate liver abscess development (Nagaraja and Chengappa, 1998; Amachawadi and Nagaraja, 2016; McDaniel et al., 2023). The overall rumenitis prevalence among CON, CV, and CVC was 26%, whereas the overall LA prevalence was 67%. Therefore, in the current experiment there was a weak relationship between rumenitis and LA development. These results suggest that alternative routes of bacterial translocation from the gastrointestinal tract to the liver occurred to develop LA. A potential route for bacterial translocation is that Salmonella in the hindgut can traverse the epithelial barrier, most likely in the small or large intestine, enter the lymphatic system, and reach the portal capillary system of the liver to initiate infection (Amachawadi et al., 2017). Additional research is necessary to understand how bacteria are translocated to the liver if not through ruminal lesions.

F. necrophorum is known to be one of the most active lysine-degrading bacteria found on the ruminal epithelium (Russell, 2006; Elwakeel et al., 2013) and can be isolated from ruminal contents of cattle fed various diets (Narayanan et al., 1997). The presence of ruminal endotoxins or F. necrophorum adhering to the ruminal epithelium with the help of outer membrane proteins could cause ruminal epithelial damage (Kumar et al., 2013). The extent of ruminal epithelial damage caused by F. necrophorum alone is unknown. Interestingly, most of the rumens scored in the present study were given a score of normal, meaning they lacked signs of rumenitis. Therefore, the lack of ruminal lesions does not align with the only route of translocation of F. necrophorum occurring via a ruminal lesion, as there were far more liver abscesses than ruminal lesions. Nonetheless, the techniques employed in the present study lack the ability to delineate the precise trajectory of F. necrophorum and S. lubbock invasion within the liver-whether it occurred through ruminal lesions or via compromised tight junctions along the lower gastrointestinal tract. These potential pathways of bacterial translocation warrant further investigation.

Liver abscesses were detected in all treatments (Table 3). No differences in LA prevalence (P=0.32) nor severity (P=0.68) were noted among treatments, even though CV and CVC decreased LA 25% and 22%, respectively, relative to CON. Likewise, number of abscesses present on each liver did not differ (P=0.31) among treatments, even though there was a 50% decrease in the number of abscesses per liver for CVC vs. CON. Of the 25 abscesses collected and sampled at the time of necropsy, CON 77.8% (n=7), CV 60.0% (n=9), and CVC 57.1% (n=8; P=0.59), 24 of abscesses yielded F. necrophorum subsp. necrophorum.

No differences among treatments were detected in colon scores (P=0.50; Table 3). A previous LA challenge study with Holstein steers also reported no difference in colon scores, although abscess prevalence was 15% of the steers in the study (McDaniel et al., 2023). Fusobacterium necrophorum subsp. necrophorum was detected within the colon tissue among all treatments, but it did not differ (P=0.49) among treatments. F. necrophorum subsp. necrophorum also was isolated from colon contents in CV, but not detected in other treatments. The colonic epithelium is a crucial physical and immunological barrier in the lower gastrointestinal tract. Increased hindgut fermentation can lead to a decrease in the pH of the large intestine, an increase in endotoxin concentration within the large intestine digesta, and subsequent damage to the hindgut epithelium (Liu et al., 2014; Ye et al., 2016). Previous studies reported that extensive hindgut fermentation can trigger inflammation, potentially caused by amines, endotoxins, or bacteria that breach an impaired intestinal barrier (Gozho et al., 2006; Crawford et al., 2007).

Detection and Quantification of F. necrophorum in Ruminal and Colonic Contents and Epithelial Tissues

In the present study, quantitative PCR analysis allowed for the determination of abundance and differentiation of F. necrophorum subsp. necrophorum and F. necrophorum subsp. funduliforme in ruminal tissue, ruminal contents, colon tissue, and colon contents. The abundance of F. necrophorum subsp. necrophorum and funduliforme determined by qPCR are presented in Table 4. For F. necrophorum subsp. necrophorum, no differences were detected in abundance within ruminal tissue or ruminal contents (P=0.68). Similarly, neither colon tissue nor its contents differed in the abundance of F. necrophorum subsp. necrophorum (P>0.47), and no F. necrophorum subsp. necrophorum was detected in ileal tissue or contents. F. necrophorum subsp. funduliforme concentration did not differ among treatments in ruminal tissue or contents (P≥0.37) or in colon tissue (P≥0.47), and F. necrophorum subsp. funduliforme was not detected in colon contents, ileal tissue, or ileal content.

TABLE 4
Abundance of Fusobacterium necrophorum subsp. necrophorum (4a)
or Fusobacterium necrophorum subsp. funduliforme (4b) identified
using quantitative polymerase chain reaction in ruminal, colonic, and
ileal tissue and contents of beef × dairy steers fed ground corn
with or without (CON) Bacillus subtilis and encapsulated butyric
acid and zinc (CV), and CV plus red clover (Trifolium pratense;
CVC) to evaluate the formation of liver abscesses

| | Treatment¹ | | | | |
|---|---|---|---|---|---|
| Tissue | CON | CV | CVC | SEM² | P-value |

Subsp. necrophorum

| | | | | | |
|---|---|---|---|---|---|
| Rumen tissue³ | 2.67 | 3.35 | 3.59 | 0.635 | 0.67 |
| Rumen | 1.43 | 1.29 | 1.88 | 0.483 | 0.68 |
| contents⁴ | | | | | |
| Colon tissue³ | 1.00 | 0.32 | 0.97 | 0.436 | 0.49 |
| Colon contents⁴ | ND | 0.24 | ND | 0.147 | 0.47 |
| Ileum Tissue³ | ND | ND | ND | — | — |
| Ileum contents⁴ | ND | ND | ND | — | — |

Subsp. funduliforme

| | | | | | |
|---|---|---|---|---|---|
| Rumen tissue³ | 1.48 | 1.92 | 0.76 | 0.572 | 0.37 |
| Rumen | 2.04 | 1.65 | 1.25 | 0.475 | 0.60 |
| contents⁴ | | | | | |
| Colon tissue³ | 0.51 | ND | 0.31 | 0.260 | 0.47 |
| Colon contents⁴ | ND | ND | ND | — | — |
| Ileum Tissue³ | ND | ND | ND | — | — |
| Ileum contents⁴ | ND | ND | ND | — | — |

ND = non-detectable.
¹CON = control diet and acidotic diet with intraruminal bacterial inoculation; CV = control diet and acidotic diet supplemented with ground corn, CLOSTAT ® Dry, and VilliTech ® with intraruminal bacterial inoculation; CVC = CV + red clover with intraruminal bacterial inoculation.
²Standard error of least squares means, n = 9 steers in CON; 15 steers in CV and 14 steers in CVC.
³Log₁₀ CFU/g of as-is tissue.
⁴Log₁₀ CFU/mL of contents.

Notably, both subspecies were detected among all treatments within the ruminal tissue and ruminal contents and also detected within the colon tissue and colon contents. The steers in our study were subjected to inoculation with F. necrophorum subsp. necrophorum, which combined with lactic acid production from a high-starch diet could increase the abundance of F. necrophorum in the ruminal contents. Fusobacterium necrophorum, a Gram-negative, anaerobic bacterium commensal in the rumen of cattle, serves as the predominant causative agent of liver abscesses in cattle (Scanlan and Hatchcock, 1983; Tan et al., 1996).

Nagaraja and Chengappa (1998) noted that once ruminal lesions occur, F. necrophorum can either create a ruminal lesion or enter the portal vein and be transported to hepatic tissue, a manifestation underscored by a numerical increase in the prevalence of subsp. necrophorum in the ruminal epithelial tissue of CV and CVC vs. CON (Table 5). The emergence of S. lubbock within liver abscesses, as evidenced by its isolation, and the postulation by Amachawadi and Nagaraja (2016) regarding the conceivable role of a leaky gut phenomenon, accentuates a secondary plausible route for the invasion of pathogenic bacteria into the hepatic portal system.

TABLE 5
Bacterial prevalence within liver abscesses collected at the time of harvest
of beef × dairy steers fed ground corn with or without (CON) Bacillus
subtilis and encapsulated butyric acid and zinc (CV), and CV + red clover
(Trifolium pratense; CVC) to evaluate the formation of liver abscesses
Liver abscess containing bacteria, %

| | Treatment¹ | | | | P- |
|---|---|---|---|---|---|
| Bacteria | CON | CV | CVC | SEM² | value |
| F. necrophorum | 77.8 | 60 | 57.1 | 0.75 | 0.59 |
| subsp. | | | | | |
| Necrophorum | | | | | |
| F. necrophorum | — | — | — | — | — |
| subsp. | | | | | |
| Funduliforme | | | | | |
| T. pyogenes | — | — | — | — | — |
| Salmonella | 44.4 | 40 | 35.7 | 0.79 | 0.81 |
| T. pyogenes and | — | — | — | — | — |
| Salmonella | | | | | |
| F. necrophorum | 44.4 | 33.3 | 28.6 | 0.81 | 0.74 |
| subsp. | | | | | |
| Necrophorum and | | | | | |
| Salmonella | | | | | |
| F. necrophorum | — | — | — | — | — |
| subsp. | | | | | |
| Necrophorum, | | | | | |
| T. pyogenes and | | | | | |
| Salmonella | | | | | |

¹CON = control diet and acidotic diet with intraruminal bacterial inoculation; CV = control diet and acidotic diet supplemented with ground corn, CLOSTAT ® Dry, and VilliTech ® with intraruminal bacterial inoculation; CVC = CV + red clover with intraruminal bacterial inoculation.
²Standard error of least squares means, n = 9 steers in CON; 15 steers in CV and 14 steers in CVC.

Fusobacterium necrophorum actively degrades proteins and amino acids, with particular emphasis on lysine obtained from feed and the ruminal epithelium (Russell, 2006; Elwakeel et al., 2013). In forage-fed steers, the population of F. necrophorum remains relatively low, generally less than 1×10⁵ per gram of ruminal contents (Tan et al., 1994c). Nonetheless, as intake of starch increases, as in the current study, the population of F. necrophorum increases, exceeding 1×10⁶ per gram of ruminal contents. This increase is associated with availability of lactate because of the introduction of the high-concentrate, acidotic diet (Coe et al., 1999; Tan et al., 1994a). The increase in starch intake and subsequent lactic acid production likely served as a substrate for the proliferation of F. necrophorum and provided a favorable environment within the rumen of the beef×dairy steers in the current study.

Bacterial Isolation from Liver Abscesses

There was no difference detected in the percentage of liver abscesses containing F. necrophorum subsp. necrophorum among treatments (P=0.59: Table 5). Moreover, F. necrophorum subsp. funduliforme and Trueperella pyogenes were not isolated in combination from any liver abscesses. No differences were detected for the percentage of liver abscesses containing S. enterica (P=0.81). One scar that was collected at the time of necropsy (1 steer in CVC) did not yield Fusobacterium subsp. necrophorum nor subsp. funduliforme but was positive for S. enterica. Moreover, no differences were observed in the proportion of liver abscesses that contained a combination of F. necrophorum subsp. necrophorum and S. enterica (P=0.74). No liver abscesses collected during the current study yielded T. pyogenes or F. necrophorum subsp. funduliforme.

The ability of F. necrophorum to migrate to and establish itself in the liver is facilitated by competitive advantages. Ruminal tissue damage can occur when these endotoxins are present or when F. necrophorum adheres to the rumen wall via the outer membrane protein (Kumar et al., 2013), and F. necrophorum is aerotolerant and exhibits remarkable oxygen tolerance (Hofstad, 1984), allowing colonization of the ruminal epithelium. The bacterium also can proliferate efficiently at the slightly basic pH commonly observed at the ruminal epithelial surface (Tan et al., 1994a) in contrast to overall reticulorumen pH in homeostatic conditions primarily ranging from 5.8 to 6.5 Nagaraja and Titgemeyer, 2007). In the current study, F. necrophorum subsp. necrophorum was present in most liver abscesses. The absence of F. necrophorum subsp. funduliforme and T. pyogenes emphasize the opportunistic ability of F. necrophorum subsp. necrophorum to outcompete other bacteria.

### Salmonella Prevalence

Initially, on d −18 only a small proportion of steers were shedding Salmonella in their feces, which did not differ among treatments (P=0.98; Table 6). Subsequently, no differences (P≥0.12) in fecal Salmonella shedding on days 2 or 38 were detected. A notable increase in Salmonella prevalence in feces was evident on d 24 (P=0.12). The increased fecal Salmonella prevalence in all treatments occurred after ruminal inoculation on day 20, which included S. enterica subsp. Lubbock. Furthermore, there were no differences in Salmonella prevalence within subiliac or mesenteric lymph nodes among treatments after harvest (P≥0.87), and Salmonella prevalence in the ileum did not differ (P=0.44) among treatments.

TABLE 6
Salmonella prevalence in fecal samples, subiliac lymph nodes, mesenteric
lymph nodes and ileum tissue from beef × dairy steers fed ground
corn with or without (CON) Bacillus subtilis and encapsulated butyric
acid and zinc (CV), and CV + red clover (Trifolium pratense;
CVC) to evaluate the formation of liver abscesses.

| | Treatment¹ | | | | |
|---|---|---|---|---|---|
| Item | CON | CV | CVC | SEM² | P-value |
| Fecal d −18, | 0.0 | 0.0 | 6.7 | 6.44 | 0.98 |
| % | | | | | |
| Fecal d 24, | 77.8 | 93.3 | 85.7 | 13.87 | 0.12 |
| % | | | | | |
| Fecal d 38, | 55.6 | 60.0 | 42.9 | 16.58 | 0.74 |
| % | | | | | |
| Subiliac | 0.0 | 6.7 | 0.0 | 19.96 | 0.97 |
| lymph | | | | | |
| nodes, % | | | | | |
| Mesenteric | 11.1 | 13.3 | 7.1 | 10.48 | 0.87 |
| lymph | | | | | |
| nodes, % | | | | | |
| Ileum, % | 0.0 | 26.7 | 7.1 | 11.42 | 0.44 |

¹CON = control diet and acidotic diet with intraruminal bacterial inoculation; CV = control diet and acidotic diet supplemented with ground corn, CLOSTAT ® dry, and VilliTech ® with intraruminal bacterial inoculation; CVC = CV + red clover with intraruminal bacterial inoculation.
²Standard error of least squares means, n = 9 steers in CON; 15 steers in CV and 14 steers in CVC.

The exact contribution of Salmonella in LA formation remains unclear, and it has only recently been isolated from liver abscesses (Amachawadi and Nagaraja, 2015). Nevertheless, Salmonella can colonize the intestines, particularly the small intestine, and stress can cause disruptions of tight junction barrier function (Boyle et al., 2006), which could allow Salmonella to invade the lymphatic system and liver (Rings, 1985). Current results indicated that Salmonella was isolated from 40% of liver abscesses, whereas previous research indicated that Salmonella was only isolated from 25% of naturally occurring abscesses (Amachawadi and Nagaraja, 2016). Similar to our results, McDaniel et al. (2023) reported 57% Salmonella prevalence in Holstein steers used in an experimental model to induce liver abscesses. Herrick et al. (2022) reported a breed effect, with Holsteins exhibiting an increased prevalence of Salmonella (40%) compared with beef breeds (29.5%). Given that McDaniel et al. (2023) reported 57% Salmonella prevalence in liver abscesses and 40% Salmonella was isolated from liver abscesses in the current study using beef×dairy steers, these results support the findings of Herrick et al. (2022) that there could be a breed effect associated with the prevalence of Salmonella within liver abscesses.

Although no statistical differences in LA were detected following supplementation, the decrease in liver abscess prevalence observed with CV could be biologically and economically impactful to the beef industry. Further research is necessary to not only determine repeatability, but to elucidate the mechanism(s) by which the combination of these supplements impact liver abscess formation. Furthermore, these data illustrate that the experimental model of acidosis and bacterial inoculation we used can be a viable model to evaluate novel strategies to mitigate LA thereby allowing producers to make informed decisions.

### Example 2

Materials and Methods: Four feedyards in the high plains region, of the United States of America, were used for this study. Yards were randomly assigned to treatments; Control, normal diet for natural cattle in the high plains region of the United States (CON, n=3,572 head); Treatment, normal diet for natural cattle in the high plains region of the U.S. and supplemented 0.5 g/head daily B. subtilis PB6 (CLOSTAT® 500, 6.60×10⁹ CFU/g, Kemin Industries, Inc., Des Moines, IA)+3 g/head daily of butyric acid (0.75 g daily) and zinc (0.3 g daily; VilliTech, Kemin Industries, Inc; CV; n=4,406 head). Anywhere between 200-500 head were harvested per treatment from 157-233 days on feed.

### Results and Discussion:

### Liver Abscess Prevalence

The CV treatment increased the prevalence of normal livers from 157-233 DOF (“DOF” as used herein refers to Days of Feed, 12 harvest days) by 6.36% and decreased the amount of abnormal livers by 9.18% compared to control (Table 7). When singling out harvest days CV increased the percentage of normal livers by 34.05% on Day 170, 28.35% on Day 205 and 15.38% on Day 226, respectively. It was observed that the combination of Bacillus subtilis PB6, butyric acid, and zinc are proving to be a useful combination in decreasing the occurrence of abnormal livers in natural fed cattle.

TABLE 7
Liver abscess prevalence from Bos Taurus influences natural
cattle supplemented with or without (CON) Bacillus subtilis
PB6 and encapsulated butyric acid and zinc (CV).

| | | Treatment¹ | | |
|---|---|---|---|---|
| Item | Days On Feed | CON | CV | %, Difference |
| Head | 157-233 | 3572 | 4406 | |
| Normal Liver, % | | 57.28 | 61.03 | 6.34 |
| Abnormal Liver, % | | 42.72 | 38.97 | −9.18 |
| Head | 157 | 304 | 304 | |
| Normal Liver, % | | 56.91 | 58.22 | 2.28 |
| Head | 164 | 304 | 379 | |
| Normal Liver, % | | 63.16 | 67.55 | 6.72 |
| Head | 170 | 342 | 266 | |
| Normal Liver, % | | 48.25 | 68.05 | 34.05 |
| Head | 178 | 304 | 380 | |
| Normal Liver, % | | 58.88 | 59.21 | 0.56 |
| Head | 184 | 266 | 380 | |
| Normal Liver, % | | 60.53 | 66.32 | 9.13 |
| Head | 197 | 304 | 379 | |
| Normal Liver, % | | 63.49 | 65.96 | 3.82 |
| Head | 199 | 304 | 380 | |
| Normal Liver, % | | 46.38 | 41.84 | −10.29 |
| Head | 205 | 304 | 380 | |
| Normal Liver, % | | 55.59 | 73.95 | 28.35 |
| Head | 212 | 304 | 380 | |
| Normal Liver, % | | 60.86 | 56.32 | −7.75 |
| Head | 219 | 304 | 456 | |
| Normal Liver, % | | 64.47 | 54.39 | −16.96 |
| Head | 226 | 228 | 456 | |
| Normal Liver, % | | 53.95 | 62.94 | 15.38 |
| Head | 233 | 304 | 266 | |
| Normal Liver, % | | 55.59 | 59.77 | 7.25 |

¹CON = control diet; CV = control diet, CLOSTAT ® 500, and VilliTECH ®

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

### REFERENCES

Albornoz R. I., J. R. Aschenbach, D. R. Barreda, and G. B. Penner. 2013. Feed restriction decreases short-chain fatty acid absorption across the reticulorumen of beef cattle independent of diet. J. Anim. Sci. 91:4730-4738. doi:10.2527/jas.2012-6223.
Allard M. W., Y. Luo, E. Strain, C. Li, C. E. Keys, I. Son, R. Stones, S. M. Muser, and E. W. Brown. 2012. High resolution clustering of Salmonella enterica serovar Montevideo strains using next-generation sequencing approach. BMC Genomics, 13:32 (18 pp). doi.org/10.1186/1471-2164-13-32.
Amachawadi, R. G., and T. G. Nagaraja. 2015. First report of anaerobic isolation of Salmonella enterica from liver abscesses of feedlot cattle. J. Clin. Microbiol. 53:3100-3101. doi:10.1128/JCM.01111-15.
Amachawadi, R. G., and T. G. Nagaraja. 2016. Liver abscesses in cattle: A review of incidence in Holsteins and of bacteriology and vaccine approaches to control in feedlot cattle. J. Anim. Sci. 94:1620-1632. doi:10.2527/jas.2015-0261.
Amachawadi, R. G., T. J. Purvis, B. V. Lubbers, J. W. Homm, C. L. Maxwell, and T. G. Nagaraja. 2017. Bacterial flora of liver abscesses in crossbred beef cattle and Holstein steers fed finishing diets with or without tylosin. J. Anim. Sci. 95:3425-3434. doi:10.2527/jas2016.1198.
Arthur, T. M., D. M. Brichta-Harhay, J. M. Bosilevac, M. N. Guerini, N. Kalchayanand, J. E. Wells, S. D. Shackelford, T. L. Wheeler, and M. Koohmaraie. 2008. Prevalence and characterization of Salmonella in bovine lymph nodes potentially destined for use in ground beef. J. Food. Prot. 71:1685-1688. doi:10.4315/0362-028x-71.8.1685.
Beauchemin, K. A., and S. M. McGinn. 2006. Methane emissions from beef cattle: Effects of fumaric acid, essential oil, and canola oil1. J. Anim. Sci. 84:1489-1496. doi:10.2527/2006.8461489x.
Broadway, R. P., J. A. Carroll, N. C. Burdick Sanchez, T. R. Callaway, S. D. Lawhon, E. V. Gart, L. K. Bryan, D. J. Nisbet, H. D. Hughes, J. F. Legako, D. L. Oconnor, J. E. Hergenreder, and P W. Rounds. Bacillus subtilis PB6 supplementation in weaned Holstein steers during an experimental Salmonella challenge. Foodborne Pathog. Dis. 17:521-528. doi:10.1089/fpd.2019.2757.
Brown, H., R. F. Bing, H. P. Grueter, J. W. McAskill, C. O. Cooley, and R. P. Rathmacher, 1975. Tylosin and chlortetracycline for the prevention of liver abscesses, improved weight gains and feed efficiency in feedlot cattle. J. Anim. Sci. 40:207-213. doi:10.2527/jas1975.402207x.
Brown T. R., and T. E. Lawrence. 2010. Association of liver abnormalities with carcass grading performance and value. J. Anim. Sci. 88:4037-4043. doi:10.2527/jas.2010-3219.
Boyle, E. C., N. F. Brown, and B. B. Finlay. 2006. Salmonella enterica serovar Typhimurium effectors SopB, SopE, SopE2 and SipA disrupt tight junction structure and function. Cell. Microbiol. 8:1946-1957. doi/10.1111/j.1462-5822.2006.00762.x
Coe, M. L., T. G. Nagaraja, Y. D. Sun, N. Wallace, E. G. Towne, K. E. Kemp, and J. P. Hutcheson. 1999. Effect of virginiamycin on ruminal fermentation in cattle during adaptation to a high concentrate diet and during an induced acidosis. J. Anim. Sci. 77:2259-2268. doi:10.2527/1999.7782259x.
Crawford, C., M. F. Sepulveda, J. Elliott, P. A. Harris, and S. R. Bailey. 2007. Dietary fructan carbohydrate increases amine production in the equine large intestine: implications for pasture-associated laminitis. J. Anim. Sci. 85:2949-2958. doi:10.2527/jas.2006-600.
Dehghani, N., M. Afsharmanesh, M. Salarmoini, and H. Ebrahimnejad. 2019. In vitro and in vivo evaluation of thyme (Thymus vulgaris) Essential Oil as an Alternative for Antibiotic in Quail Diet. J. Anim. Sci. doi:10.1093/jas/skz179. Available from: https://academic.oup.com/jas/advance-article/doi/10.1093/jas/skz179/5497532
Elwakeel, E. A., R. G. Amachawadi, A. M. Nour, M. E. A. Nasser, T. G. Nagaraja, and E. C. Titgemeyer. 2013. In vitro degradation of lysine by ruminal fluid-based fermentations and by Fusobacterium necrophorum. J. Dairy Sci. 96:495-505. doi:10.3168/jds.2012-5810.
Flythe M., and I. Kagan. 2010. Antimicrobial effect of red clover (Trifolium pratense) phenolic extract on the ruminal hyper ammonia-producing bacterium, Clostridium sticklandii. Curr Microbiol. 61:125-31. 10.1007/s00284-010-9586-5.
Global Trade Atlas. 2017. https://www.gtis.com/gta/. (Accessed 20 Jun. 2023).
Gozho, G., D. Krause, and J. Plaizier. 2006. Rumen lipopolysaccharide and inflammation during grain adaptation and subacute ruminal acidosis in steers. J. Dairy Sci. 89:4404-4413. doi:10.3168/jds.S0022-0302 (06) 72487-0.
Harlow B. E., M. D. Flythe, I. A. Kagan, G. E. Aiken. 2017. Biochanin A (an isoflavone produced by red clover) promotes weight gain of steers grazed in mixed grass pastures and fed dried-distillers' grains. Crop Sci. 57:506-514. doi:10.2135/cropsci2016.07.0590.
Harlow B. E., M. D. Flythe, G. E. Aiken. 2018 Biochanin A improves fibre fermentation by cellulolytic bacteria. J Appl. Microbiol. 124:58-66. doi10.1111/jam.13632.
Herrick, R. T., C. L. Rogers, T. J. McEvers, R. G. Amachawadi, T. G. Nagaraja, C. L. Maxwell, J. B. Reinbold, and T. E. Lawrence. 2022. Exploratory observational quantification of liver abscess incidence, specific to region and cattle type, and their associations to viscera value and bacterial flora. Appl. Anim. Sci. 38:170-182. doi:10.15232/aas.2021-02228.
Hofstad, T. 1984. Pathogenicity of anaerobic Gram-negative rods: possible mechanisms. Rev. Infect. Dis. 6:189-199. doi:10.1093/clinids/6.2.189.
Ko, W-C., L.-H Lin, H-Y. Shen, Lai, C-Y Lai, C-M. Chen, and C. H Shih. 2011. Biochanin A, a phytoestrogenic isoflavone with selective inhibition of phosphodiesterase 4, suppresses ovalbumin-induced airway hyperresponsiveness. Evidence-Based Complementary and Alternative Medicine 1:13. doi:10.1155/2011/635058.
Kumar, A., E. Gart, T. G. Nagaraja, and S. Narayanan. 2013. Adhesion of Fusobacterium necrophorum to bovine endothelial cells is mediated by outer membrane proteins. Vet. Microbiol. 162:813-818. doi:10.1016/j.vetmic.2012.10.022
Liu, J. H., T. T. Xu, W. Y. Zhu, and S. Y. Mao. 2014. High-grain feeding alters caecal bacterial microbiota composition and fermentation and results in caecal mucosal injury in goats. Br. J. Nutr. 112:416-427. doi:10.1017/S0007114514000993.
Mansfield, L. S., and J. F. Urban. 1996. The pathogenesis of necrotic proliferative colitis in swine is linked to whipworm induced suppression of mucosal immunity to resident bacteria. Vet. Immunol. Immunopath. 50:1-17. doi:10.1016/0165-2427 (95) 05482-0.
McDaniel, Z. S. K. E. Hales, H. Salih, A. Deters, X. Shi, T. G. Nagaraja, T. E. Lawrence, T. C. Tennant, R. G. Amachawadi, J. A. Carroll, N. C. Burdick Sanchez, M. L. Galyean, T. M. Smock, M. A. Ballou, V. S. Machado, E. Davis, P. R. Broadway. 2023. Development of an experimental model to induce liver abscesses in steers using an acidotic diet challenge and intraruminal bacterial inoculation. J. Anim. Sci. In Press.
Nagaraja, T. G., and M. M. Chengappa. 1998. Liver abscesses in feedlot cattle: a review. J. Anim. Sci. 76:287-298. doi:10.2527/1998.761287x.
Nagaraja T. G, E. C. Titgemeyer. 2007. Ruminal acidosis in beef cattle: The current microbiological and nutritional outlook. J. Dairy Sci. 90:E17-E38. doi:10.3168/jds.2006-478.
Narayanan, S. K., T. G. Nagaraja, O. Okwumabua, J. Staats, M. M. Chengappa, R. D. Oberst. 1997. Ribotyping to compare Fusobacterium necrophorum isolates from bovine liver abscesses, ruminal walls, and ruminal contents. Appl. Environ. Microbiol. 63:4671-4678. doi:10.1128/aem.63.12.4671-4678.1997.
NASEM. 2016. Nutrient requirements of beef cattle. (8th rev. ed.), Natl. Acad. Press, Washington, DC, USA.
Ohata, A., M. Usami, M. Miyoshi. 2005. Short-chain fatty acids alter tight junction permeability in intestinal monolayer cells via lipoxygenase activation. Nutrition 21:838-847. doi:10.1016/j.nut.2004.12.004.
Owens, F. N., D. S. Secrist, W. J. Hill, and D. R. Gill. 1998. Acidosis in cattle: a review. J. Anim. Sci. 76:275-286. doi:10.2527/1998.761275x.
Rezac, D. J., D. U. Thomson, M. G. Siemens, F. L. Prouty, C. D. Reinhardt, and S. J. Bartley. 2014. A survey of gross pathologic conditions in cull cows at slaughter in the Great Lakes region of the United States. J. Dairy Sci. 97:4227-4235. doi:10.3168/jds.2013-7636.
Rings, M. D. 1985. Salmonellosis in calves. Vet. Clin. North Am. Food Anim., 1:529-539. doi.org/10.1016/S0749-0720 (15) 31301-3.
Rode, L. M., T. A. Mcallister, K. A. Beauchemin, D. P. Morgavi, V. L. Nsereko, W. Z. Yang, A. D. Iwaasa, and Y. Wang. 2002. Enzymes as direct-feed additives for ruminants. In: R. Renaville and A. Burny, editors. Biotechnology in Animal Husbandry. Vol. 5. Kluwer Academic Publishers, Dordrecht. p. 301-332. Available from: http://link.springer.com/10.1007/0-306-46887-5_17
Russell, J. B. 2006. Factors affecting lysine degradation by ruminal fusobacteria. FEMS Microbiol. Ecol. 56:18-24. doi:10.1111/j.1574-6941.2006.00041.x.
Sanz-Fernandez M. V., J. B. Daniel, D. J. Seymour, S. K. Kvidera, Z. Bester, J. Doelman, and J. Martín-Tereso. 2020. Targeting the hindgut to improve health and performance in Cattle. Animals. 10 (10): 1817. doi/10.3390/ani10101817.
Scanlan, C. M., and T. L. Hathcock. 1983. Bovine rumenitis-liver abscess complex: a bacteriological review. Cornell Vet. 73:288-297.
Smith, Z. K., P. R. Broadway, K. R. Underwood, W. C. Rusche, J. A. Walker, N. C. Burdick-Sanchez, J. A. Carroll, D. Lafleur, and J. E. Hergenreder. 2021. Evaluation of Bacillus subtilis PB6 on feedlot phase growth performance, efficiency of dietary net energy utilization, and fecal and subiliac lymph node Salmonella prevalence in spring placement yearling beef steers fed in southeastern South Dakota. Transl. Anim Sci. 5: txab002. doi:10.1093/tas/txab002.
Smock T. M., K. L. Samuelson, J. E. Hergenreder, P. W. Rounds, and J. T. Richeson. 2020. Effects of Bacillus subtilis PB6 and/or chromium propionate supplementation on clinical health, growth performance, and carcass traits of high-risk cattle during the feedlot receiving and finishing periods. Transl Anim Sci. 4: txaa163 doi:10.1093/tas/txaa163.
Step, D. L., C. R. Krehbiel, H. A. Depra, J. J. Cranston, R. W. Fulton, J. G. Kirkpatrick, D. R.Gill, M. E. Payton, M. A. Montelongo, and A. W. Confer. 2008. Effects of commingling beef calves from different sources and weaning protocols during a forty-two-day receiving period on performance and bovine respiratory disease. J. Anim. Sci. 86:3146-3158. doi:10.2527/jas.2008-0883.
Thompson, P. N., A. Stone, and W. A. Schultheiss. 2006. Use of treatment records and lung lesion scoring to estimate the effect of respiratory disease on growth during early and late finishing periods in South African feedlot cattle. J. Anim. Sci. 84:488-498. doi:10.2527/2006.842488x.
Tan, Z. L., K. F. Lechtenberg, T. G. Nagaraja, M. M. Chengappa, and R. T. Brandt, Jr. 1994a. Serum neutralizing antibodies against Fusobacterium necrophorum leukotoxin in cattle with experimentally induced or naturally developed hepatic abscesses. J. Anim. Sci. 72:502-508. doi:10.2527/1994.722502x.
Tan, Z. L., T. G. Nagaraja, and M. M. Chengappa. 1994b. Biochemical and biological characterization of ruminal Fusobacterium necrophorum. FEMS Microbiol. Lett. 120:81-86. doi:10.1111/j.1574-6968.1994.tb07011.x.
Tan, Z. L., T. G. Nagaraja, and M. M. Chengappa. 1994c. Selective enumeration of Fusobacterium necrophorum from the bovine rumen. Appl. Environ. Microbiol. 60:1387-1389. doi/10.1128/aem.60.4.1387-1389.1994.
Tan, Z. L., T. G. Nagaraja, and M. M. Chengappa. 1996. Fusobacterium necrophorum infections: virulence factors, pathogenic mechanism and control measures. Vet. Res. Commun. 20:113-140. doi:10.1007/BF00385634.
Tennant, T. C., S. E. Ives, L. G. Harper, D. G. Renter, and T. E. Lawrence. 2014. Comparison of tulathromycin and tilmicosin on the prevalence and severity of bovine respiratory disease in feedlot cattle in association with feedlot performance, carcass characteristics, and economic factors. J. Anim. Sci. 92:5203-5213. doi:10.2527/jas.2014-7814.
Veterinary Feed Directive. 2015. Fed. Regist. Available from: https://www.federalregister.gov/documents/2015/06/03/2015-13393/veterinary-feed-directive
Weinroth, M. D., Martin, J., Doster, E., Geornaras, I., Parker, J., Carlson, C., Metcalf, J., Morley, P., and Belk, K. 2019. Investigation of tylosin in feed of feedlot cattle and effects on liver abscess prevalence, and fecal and soil microbiomes and resistomes. J. Anim. Sci. 97 (11): 4567-4578. doi:10.1093/jas/skz306
Wilson, B. K., D. L. Step, C. L. Maxwell, J. J. Wagner, C. J. Richards, and C. R. Krehbiel. 2015. Evaluation of multiple ancillary therapies used in combination with an antimicrobial in newly received high-risk calves treated for bovine respiratory disease. J. Anim. Sci. 93:3661-3674. doi:10.2527/jas.2015-9023.
Ye, H. M., J. H. Liu, P. F. Feng, W. Y. Zhu, and S. Y. Mao. 2016. Grain-rich diets altered the colonic fermentation and mucosa-associated bacterial communities and induced mucosal injuries in goats. Sci. Rep. 6:20329. doi:10.1038/srep20329.
Zhang S., J. R. Aschenbach, D. R. Barreda, and G. B. Penner. 2013. Recovery of absorptive function of the reticulo-rumen and total tract barrier function in beef cattle after short-term feed restriction. J. Anim. Sci. 91:1696-1706. doi:10.2527/jas.2012-5774.
Zhang, F., T. G. Nagaraja, D. George, G. C. Stewart. 2006. The two major subspecies of Fusobacterium necrophorum have distinct leukotoxin operon promoter regions. Vet. Microbiol. 112:73-78. doi:10.1016/j.vetmic.2005.10.003.

1. A method of decreasing incidence of liver abscess in cattle on feedlots comprising the steps of orally administering to the cattle a supplement that contains an effective amount of Bacillus subtilis PB6, butyric acid and zinc for at least 25 days prior to harvest.

2. The method of claim 1, wherein the supplement further comprises red clover or an extract of red clover.

3. The method of claim 1, wherein the supplement is administered daily for at least 35 days prior to harvest.

4. The method of claim 1, wherein the supplement is administered daily for at least 50 days prior to harvest.

5. The method of claim 1, wherein the supplement is administered daily for at least 75 days prior to harvest.

6. The method of claim 1, wherein the supplement is mixed into animal diet.

7. The method of claim 6, wherein the supplement is top dressed and mixed with the diet at the time of feeding.

8. A method of decreasing severity of liver abscess in cattle on feedlots comprising the steps of administering a feed additive to the cattle comprising an effective amount of Bacillus subtilis PB6, butyric acid and zinc for at least 25 days prior to harvest.

9. The method of claim 8, wherein the additive further comprises red clover or an extract of red clover.

10. The method of claim 8, wherein the additive is administered daily for at least 35 days prior to harvest.

11. The method of claim 8, wherein the additive is administered daily for at least 50 days prior to harvest.

12. The method of claim 8, wherein the additive is administered daily for at least 75 days prior to harvest.

13. The method of claim 8, wherein the additive is mixed into animal diet.

14. The method of claim 13, wherein the additive is top dressed and mixed with the diet at the time of feeding.

15. A method of improving scoring of a beef carcass at harvest comprising the steps of orally administering an effective amount of an animal feed supplement containing Bacillus subtilis PB6, butyric acid and zinc to the cattle for at least 25 days prior to harvest.

16. The method of claim 15, wherein the supplement further comprises red clover or an extract of red clover.

17. The method of claim 15, wherein the supplement is administered daily for at least 50 days prior to harvest.

18. The method of claim 15, wherein the supplement is administered daily for at least 75 days prior to harvest.

19. The method of claim 15, wherein the supplement is mixed into animal diet.

20. The method of claim 19, wherein the supplement is top dressed and mixed with the diet at the time of feeding.
